(19) United States
(12) Patent Application Publication
(10) Pub. No.: US 2021/0060325 A1
(43) Pub. Date: Mar. 4, 2021

(54) DEVICE AND METHOD FOR POWERING TATTOO DEVICE WITH VOICE CONTROL

(71) Applicant: LONG XIAO, Scarborough (CA)

(72) Inventor: LONG XIAO, Scarborough (CA)

(21) Appl. No.: 16/555,887

(22) Filed: Aug. 29, 2019

(51) Int. Cl. A61M 37/00; G06F 3/16; G10L 15/00

(52) U.S. Cl. CPC A61M 37/0076; G06F 3/167; A61M 2205/80; A61M 2205/3365; G10L 15/00

(57) ABSTRACT

A system for powering/controlling tattoo machine comprises a power supply for driving a commutatorless direct-current motor. The power supply comprises a motor driver and a connector for applying variable power to the motor to adjust its speed. A controller integrated with or connected to the power supply for controlling the power supplied to the motor to control its speed comprises microphone for detecting voice command, and processor for processing the voice command and for controlling the power output of the motor driver based on the voice command. A foot pedal comprises a switch connected to the controller. The switch is switchable between on-state and off-state. The controller is configured and operable to receive and respond to the voice command when the switch is on, but to ignore the voice command when the switch is off.

### FIELD

The present disclosure relates generally to devices and methods for controlling operation of tattoo machines or devices, particularly to systems, devices and methods for speed control and voice control of tattoo devices.

### BACKGROUND

A tattooing system typically includes a tattoo machine or device for applying ink to skin with needles and a power supply for supplying power to the tattoo machine or device. In some tattooing systems, a foot pedal may be connected to the power supply and used as a switch for starting and stopping the tattoo device. In operation, the tattoo device is hand-held by an operator and manipulated to contact a subject's skin with the needles. The needles are actuated by a needle actuator in the tattoo device to move up and down repeatedly at a selected speed or frequency, thereby repeatedly puncturing or penetrating the skin. Ink is stored in the tattoo device or otherwise applied to the needles so that the ink can flow along the needles and being applied to the punctured skin.

Typical tattoo devices may be operated at a drive frequency of 50-200 Hz, such as about 80 Hz to about 150 Hz. The operator often needs to adjust the operating frequency of the tattoo device during a tattoo session to achieve different results and visual effects. For example, when the operator is drawing a pattern of relatively low complexity or with thicker lines, the operator may move the tattoo device relatively quickly across the skin, and the tattoo device may be operated at a relatively high frequency, such as 120-150 Hz, to transfer more ink to the skin in unit time to avoid unintended broken lines. In comparison, when the operator is moving the tattoo device relatively slowly across the skin, or when the operator is holding the tattoo device stationary, the tattoo device's operating frequency may be relatively low, such as 60-90 Hz, to reduce unnecessary damage to the subject's skin and to avoid overflow of the ink. As another example, the operator may operate the tattoo device at a higher frequency when creating thin outlines, and at a lower frequency when applying shading in an area.

The operating frequency of the tattoo device may be adjusted by changing the voltage supplied by the power supply to the tattoo device. For example, in some tattoo devices, the power supply may be adjusted using a knob or dial, which can be manually operated by the operator. The operator may need to suspend tattooing operation, change the power supply output voltage by turning the knob or dial by hand to a position corresponding to the desired voltage (frequency), and then resume tattooing operation at the selected frequency. During a tattoo session, the operator's hand(s) can contact bodily fluids, such as blood or serum, from the punctured skin of the subject. For safety reasons, the operator typically wears protective gloves during a tattoo session. It is inconvenient if the operator needs to remove and change gloves from time to time in order to adjust the power supply's output voltage without transferring pathogens between the subject and the tattooing system. A hands-free control system can avoid this problem.

Known hands-free control systems for tattoo devices include, for example, the devices disclosed in U.S. Pat. No. 9,931,185 to Gagliano, which uses a foot-operated power varying pedal to vary the output voltage of the power supply of a tattoo device. The output voltage of the power supply is varied based on an amount of the pressure being applied to the pedal. However, a drawback of such a system is that the operator must use his or her foot to continuously and actively control the power supply output. The foot must remain in contact with the pedal during operation, and the operator needs to continuously monitor the output level of the power supply to ensure the tattoo device is operating at the desired frequency.

A tattoo machine may include a mini-motor for driving the needle motion, where rotary motion of the motor axle is converted to reciprocal linear motion of the needle shaft by mechanical transmission. When the needle shaft completes one reciprocating cycle for each revolution (rotation) of the motor axle, the motor speed (Sₘ), typically measured in revolution per minute (RPM), can be used to calculate the reciprocal (operating) frequency of the needle shaft, which can be expressed in Hertz (Hz, one cycle per second). For example, 60 RPM=1 Hz. In some conventional DC mini-motors, the motor speed is adjusted by regulating the applied voltage. The operator can typically set the voltage to a selected value, and the device may show the actual voltage that is being applied. Depending on the particular type and operation of the motor, the motor speed increase per unit voltage increase can be different, so for different motors or even the same motor under different operation conditions (e.g. different loads), the correlation between motor speed and applied voltage can be different. Consequently, some commercially available tattoo machines use open-loop control in their power supplies. These power supplies are adjusted based on set voltage levels and do not provide direct settings for motor speed or frequency. As a result, during operation the operator would set or adjust the voltage level without knowing the actual or precise motor speed or needle frequency, and is not or adjust the motor speed directly. The operator may assume the motor speed or frequency is proportional to the voltage level, but as noted above the motor speed may be different for the same voltage setting depending on the particular tattoo device used and the load at the time as well as some other factors.

### SUMMARY

It is therefore still desirable to provide improved hands-free control of tattoo devices. It is also desirable for the operator of a tattoo device to be able to set the speed of the motor that drives the needle movement more precisely and more conveniently.

An aspect of the present disclosure thus relates to voice control of tattoo devices through their power supplies.

In an aspect of the present disclosure, there is provided a power supply and control system for a tattoo machine, wherein the tattoo machine comprises an electrical motor for actuating a needle attached to the machine. The system comprises a power supply for supplying power to drive a commutatorless direct-current motor, the power supply comprising a motor driver for providing a variable power output and a connector for connection with the commutatorless direct-current motor to apply the variable power output to the motor to adjust a speed of the motor; a controller integrated with or connected to the power supply for controlling the power supplied to the motor to control the speed of the motor. The controller comprises a microphone for detecting a voice command, and a processor programmed to process the voice command and provide an input to the motor driver to control the power output of the motor driver based on the voice command. The system also comprises a foot pedal comprising a switch connected to the controller. The switch is switchable between an on-state and an off-state when the foot pedal is pressed by a foot. The controller is configured and operable to receive and respond to the voice command when the switch is in the on-state, but to ignore the voice command when the switch is in the off-state.

In the power supply and control system described in the preceding paragraph, the commutatorless direct-current motor may be a sensorless motor and the motor driver may comprise a closed-loop speed control circuit. The system may further comprises a cable for connecting the motor driver to the motor, wherein the motor comprises three windings, the motor driver comprises at least three output connectors, and the cable comprises at least three wires for connecting respective ones of the at least three output connectors of the motor driver to corresponding ones of the three windings of the motor. The switch may be a toggle switch. The controller may comprise a speech recognition chip configured to perform speech recognition locally. The processor may be programed to control one or more operation parameters of the tattoo machine, and the voice command comprises a command to adjust or set each one of the one or more operation parameters, the one or more operation parameters comprising the speed of the motor.

The processor may be programed to adjust or set a plurality of operation parameters of the motor in a time period in response to a single voice command.

The voice command may comprise a command to increase the speed of the motor, a command to decrease the speed, and a command to set the speed at a value represented by the voice command. The controller may comprise a processor-readable storage media storing thereon a plurality of pre-defined commands, and the controller may be configured to determine if the voice command matches any of the stored commands and to execute the voice command in response to determining a match. The foot pedal may be further configured to selectively activate or deactivate the tattoo machine or the motor. The controller may be programmed to select an operation mode of the foot pedal, wherein the foot pedal is operable in a first mode to activate and deactivate detection of the voice command, and operable in a second mode to selectively activate and deactivate the tattoo machine or the motor. The controller may be configured to communicate wirelessly with a portable or mobile input device. The portable or mobile input device may be a wearable device. The mobile input device may comprise a microphone or a smart watch. The mobile input device may comprise a display and an input interface.

In another aspect of the disclosure, there is provided a tattoo machine comprising a needle actuator comprising a commutatorless direct-current motor, and a power supply and control system described herein, which is operably connected to the needle actuator.

In a further aspect of the disclosure, there is provided a tattoo device comprising a needle actuator comprising a commutatorless direct-current motor; and a controller connected for controlling operation of the needle actuator. The controller comprises a motor driver connected to the motor for measuring and controlling a speed of the motor; an acoustic sensor for detecting voice commands uttered by a user, wherein the voice commands comprise a command to set the speed of the motor at a value indicated by the command, and a processor programmed to process received voice commands and provide an input to the motor driver to control operation of the needle actuator based on the received voice commands. The controller is configured and operable to respond to the command to set the speed of the motor at the value indicated by the command. The needle actuator may comprise a sensorless motor, and the motor driver comprises a closed-loop speed control circuit. The controller may comprise a speech recognition chip configured to perform speech recognition locally.

As can be understood, it may be beneficial to know or be able to directly set or adjust the motor speed/frequency to a desired setting. Thus, in an embodiment, a tattoo machine may include a brushless DC motor, and a brushless DC motor power supply configured to provide power to the motor and display the motor speed in either RPM or Hz in real time. A closed-loop speed control circuit may be used to control operation of the brushless DC motor, and allow accurate calculation and display of the motor speed or operation frequency.

With a brushless DC motor, it is possible to provide closed-loop control of the operation and motor speed of the motor, as can be understood by those skilled in the art. It is also possible to provide accurate control and reading/display of the motor speed.

Known and commercially available brushless DC motors may be used in an embodiment disclosed herein. For example, the phase windings in such a motor may be sequentially energized at appropriate times to produce a rotating magnetic field relative to a permanent magnet rotor. The timing of this sequential energization is a function of the location of the permanent magnetic rotor with respect to the particular phase winding that is to be energized. Various techniques and devices may be used to sense the position of the permanent magnet rotor relative to the phase windings. For example, optical sensors and Hall effect devices can feed a position signal to a switching logic that selectively switches power on and off to the respective phase windings. However, such sensing devices add cost and complexity to the system, and may moreover require maintenance from time to time to assure continued proper operation.

In an embodiment of a hand-held tattoo device, the motor or power supply control unit may be separated or separately provided, from the motor. The control unit or control circuit may be connected to the motor or the hand-held device by a flexible cable, which includes a power supply line to supply power (voltage) to the motor. Such a configuration can reduce the size and weight of the hand-held tattoo device, in which the motor is located. With the reduced size and weight and a flexible connection, the hand-held device can be easier to operate and manipulate.

In comparison, if a brushless DC motor with an integrated position sensor is used, the cable connecting the controller to the motor would need to include power supply lines to respective phase windings in the motor and signal lines for transmitting data and control signals. In such a case, the cable will need to be sufficiently thick to accommodate the different lines and would be less flexible. With such a cable and the added weight and size to accommodate the sensor in the hand-held device, the device is less convenient to use.

In an embodiment of tattoo device with a sensorless brushless direct-current (BLDC) motor, a control circuit for the sensorless brushless DC motor may include a controller for receiving a sensed back electromotive force (BEMF) generated by each of a plurality of phase windings of the motor, and these BEMF signals can be used to determine a rotor position of the motor and control motor speed.

Other aspects, features, and embodiments of the present disclosure will become apparent to those of ordinary skill in the art upon review of the following description of specific embodiments in conjunction with the accompanying figures.

### BRIEF DESCRIPTION OF THE DRAWINGS

In the figures, which illustrate, by way of example only, embodiments of the present disclosure:

FIG. 1 is a schematic diagram illustrating a voice control system for a tattoo device, according to an embodiment of the present disclosure;

FIG. 2 is schematic diagram illustrating a variant of the system of FIG. 1;

FIG. 3 is a schematic diagram illustrating the power supply module in the system of FIG. 1;

FIGS. 4A-4C are schematic diagrams illustrating different embodiments of the mobile device in the system of FIG. 1;

FIG. 5 is a flowchart illustrating a method of operating the system of FIG. 1 or FIG. 2;

FIGS. 6A and 6B are schematic diagrams of a pouch for storing the mobile device shown in FIG. 1; and

FIG. 6C is a schematic diagram of an embodiment of the mobile device of FIG. 1.

### DETAILED DESCRIPTION

In overview, in selected embodiments of the present disclosure a tattoo device is controlled with a voice control system which is configured to receive and recognize one or more voice commands uttered by an operator of the tattoo device, and to adjust an operation parameter of the tattoo device, such as the needle oscillating frequency, based on the voice command. A foot pedal switch is provided to activate the voice control system when the operator is uttering voice commands to adjust the operation parameter. Further, a power supply integrated with or connected to a speed control system is provided to control the motor speed based on the voice commands, with a closed-loop speed control circuit, so that the power supply and speed control system can be used to control different tattoo devices with different motors, particularly sensorless brushless direct-current motors, with consistent speed control results.

Conveniently, the operator can reliably control the operation parameter of the tattoo device during a tattoo session without using his or her hands.

Further, in various embodiments disclosed herein it is also possible to avoid cross-contamination, unintentional adjustment of the operation parameter, and other drawbacks may be avoided.

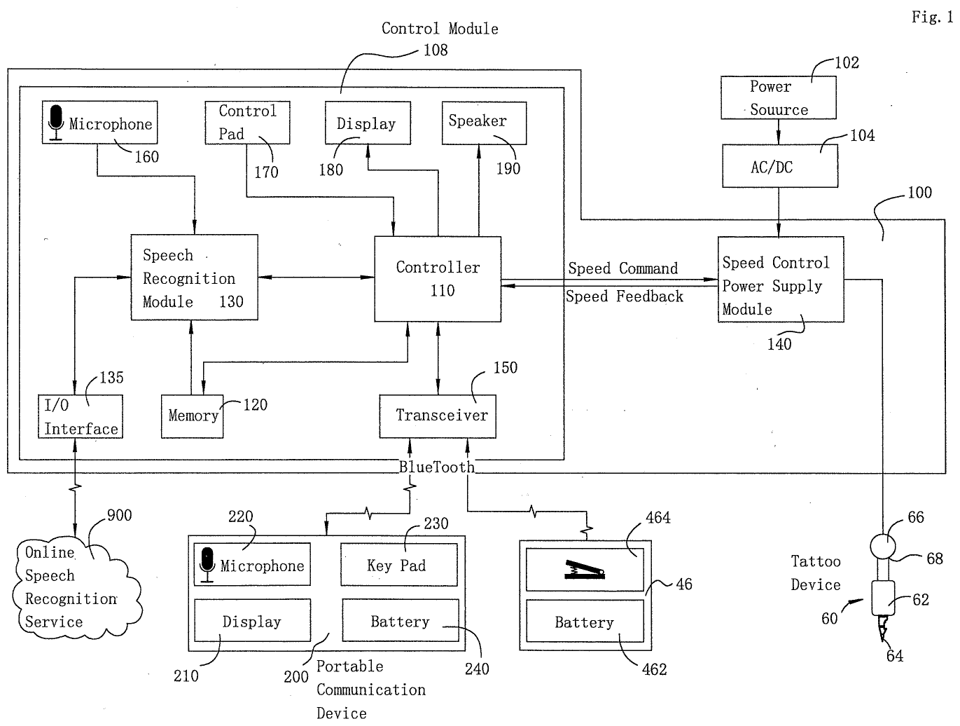

A specific embodiment relates to a voice control system 100 for a tattoo device 60, as illustrated in FIG. 1.

As depicted in FIG. 1, a tattoo device 60 (also referred to as tattoo machine) is connected to control system 100. System 100 includes a speed control power supply module 140 and a control module 108, and is connected to a power source 102 through an AC/DC converter 104.

Speed control Power supply module 140 is connected to and supplies electrical power to tattoo device 60, and may include a speed control driver circuit for alternatively providing a three-phase variable power output (e.g. variable output voltage or a pulsed voltage with a variable pulse duty ratio) for controlling the operation speed or frequency of tattoo device 60. The output voltage or pulse duty ratio may be varied based on a set voltage value, or a set motor speed or frequency, as will be further discussed below.

Power supply 140 is connected to, or has, an electrical power source. The power source can be an alternating-current (AC) power source, such as a wall power outlet (not explicitly shown), or a DC power source, such as a battery (not explicitly shown). The power source may be housed in the power supply 140, or separately provided and connected to power supply 140 such as by a cable with a plug (not shown).

As can be appreciated, tattoo device 60 includes a needle module with a bundle of needles, a needle actuator, and a handle for connecting the needle module to the needle actuator. The needle actuator typically includes a motor for reciprocally actuating the needles in the tattoo device.

When the tattoo device 60 is operated with a brushless DC (BLDC) motor, a specifically configured DC power source (see further description below) may be conveniently used. Alternatively, the AC/DC converter 104 may be used to convert the electrical current from an AC power source to a DC current. Power supply 140 may have a build-in AC/DC conversion circuit and may be configured to take AC input and provide DC output.

BLDC motors are sometimes also referred to as BL motor, commutatorless DC motor, commutatorless motor, electronically commutated motor (ECM or EC motor), or synchronous DC motors. BLDC may also be referred to as DCBL. These terms are used interchangeably herein. The BLDC or commutatorless motors used in embodiments described herein are variable speed motors.

As depicted, power supply 140 may rely on the power source 102 for the control system 100 to provide the power input.

Power supply 140 may include input/output connectors for receiving and providing powers, and input/output connectors for receiving and outputting analog or digital data or signals, as needed or desired (not explicitly shown).

Power supply 140 is configured and adapted to provide electrical voltage to tattoo device 60 at a set level, which is set based on a selected voltage or motor speed or frequency as will be further described below.

For example, the speed control module of power supply 140 may be connected to, and receive/send signals from/to, controller 110, which may include a microcontroller, or any suitable processor or microprocessor.

Controller 110 is connected to and in communication with memory 120 and a Speech Recognition module 130. Speech recognition module 130 and memory 120 may be in communication as depicted for transferring data therebeween and reading data from or storing data in memory 120.

Speech recognition module 130 is connected with an audio input/output (I/O) device such as a microphone 160 for receiving voice commands from a user of the system or the operator of tattoo device 60.

Speech recognition module 130 may include a circuit and may be programed to recognize the voice commands uttered by the operator or user. Alternatively, speech recognition module 130 may be connected to an online speech recognition service 900 through an I/O interface 135 such as a WiFi interface or cell phone or data I/O interface (such as modem or router, not shown). For example, speech recognition module 130 may include an LD3320 automatic speech recognition (ASR) chip.

In selected embodiments, for offline speech recognition, a chip model AI ASR CHIP CI1006 provided by Chipintelli may be used in speech recognition module 130. According to Chipintelli, CI1006 is based on a Brain Neural Network Processing Unit (BNPU) and can fully support highly performed Deep Neural Network (DNN) computing, integrates application processing core and other control interfaces, and can support local speech recognition and controlling functions with high accuracy, multi-language and large scale of sentences.

A voice or speech recognition module such as WTK6900B01 provided by Waytronic Electronic may also be used. The WTK6900B01 speech recognition module can identify pre-defined vocabulary entries, and provide speaker-independent recognition. The recognized vocabulary entries may be modified by the user. The desired command vocabularies can be stored in a memory storage device, such as WTK6900A-24SS voice chip storage, also provided by Waytronic Electronic, or an external storage device such as a serial peripheral interface (SPI) flash memory card.

When the speech recognition process involves communication with a remote speech recognition service such as provided by an online server or a cloud engine, a different circuit chip or processor model may be used. For example, M18516 chipset provided by MediaTek may be used to access the online or cloud speech recognition services provided by Google.

Controller 110 may also be connected to a control input device such as control pad 170 or a similar I/O device to receive input or feedback from the operator or another user. Controller 110 is further connected to one or more output devices such as a display device 180 (or monitor) and an audio output device 190 such as a speaker.

Control module 108 may include additional I/O interfaces for different communication channels. For example, as depicted, the control module may include a wireless transceiver (for example for any one or more of cellular, WiFi, BLOOTH™, or infrared connection).

Through the wireless communication, Control module 108 may be connected to a portable communication device 200 such as a wearable device or a remote control. Device 200 may be a smart watch, or a similar smart device that can be worn on a user's wrist, and may itself include a display screen 210, a microphone 220, a control pad 230 with control keys or buttons, and a battery 240. Device 200 may be used to input commands including voice commands, data and feedback into control module 100 and to review operational parameters including motor speed or frequency.

Alternatively, a wireless connection between two communicating devices can be replaced with a wired connection.

In some embodiments, Device 200 may be configured to execute limited and simple functions or the basic functions for inputting the speed control command or displaying the actual speed. In some embodiments, it may be convenient that device 200 is small in size and be easily operated and used. In such cases, control module 108 may be used to perform further and more complicated setup and configuration functions.

As depicted, power supply 140 and the control module 108 may be integrated and provided as a single unit or housed in the same housing (not shown).

System 100 includes a foot pedal 46, which in turn may include an electrical foot switch 464 and optionally, such as when the foot switch is a wireless device, a battery 462 for operating the foot switch. Alternatively, foot pedal 46 may include a mechanical switch.

In an embodiment, most components of system 100 including control module 108 and power supply 140 may be integrated and housed in the same housing such as a control box (not shown), in which case foot pedal 46 may be connected to the control box through a connection cable (not shown) or through a wireless connection.

The operation of the foot pedal 46 and the control system 100 will be discussed in further detail below.

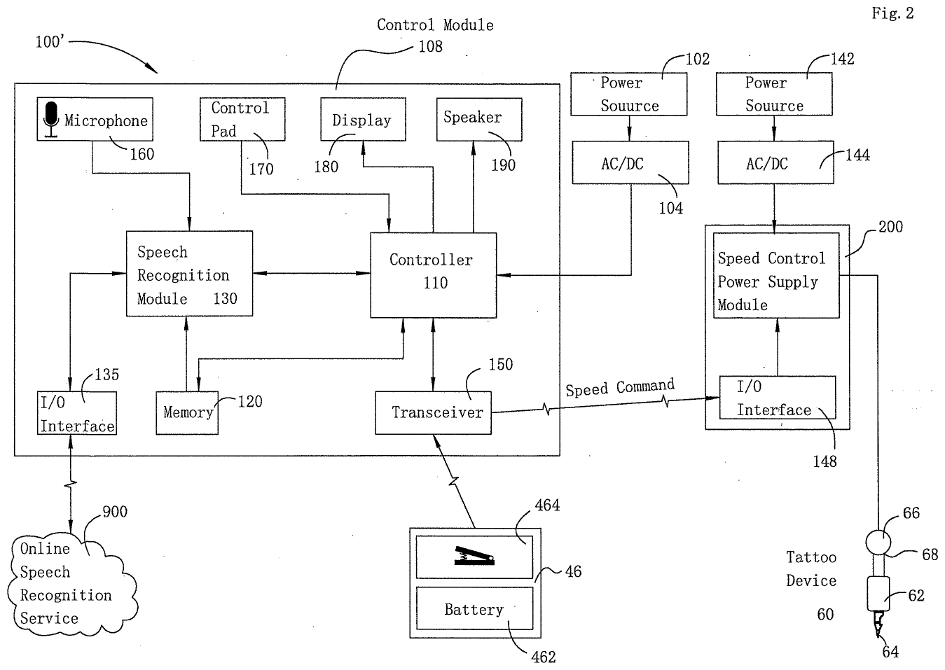

FIG. 2 illustrates an alternative, modified system 100′, which is similar to but differs from system 100 in the following aspects.

In system 100′, power supply 140 and control module 108 are separately provided in different units and may be housed in different housings. A wireless or wired I/O interface 148 such as a wireless transceiver or receiver may be provided in power supply 140 for wired or wireless communication with control module 108. In particular, power supply 140 may be configured to receive a speed setting input from the control module 108 through the I/O interface and provide an output based on the received speed setting input.

In the embodiment as depicted in FIG. 2, a separate power source 142 and AC/DC converter 144 may be provided for providing input power to power supply 140.

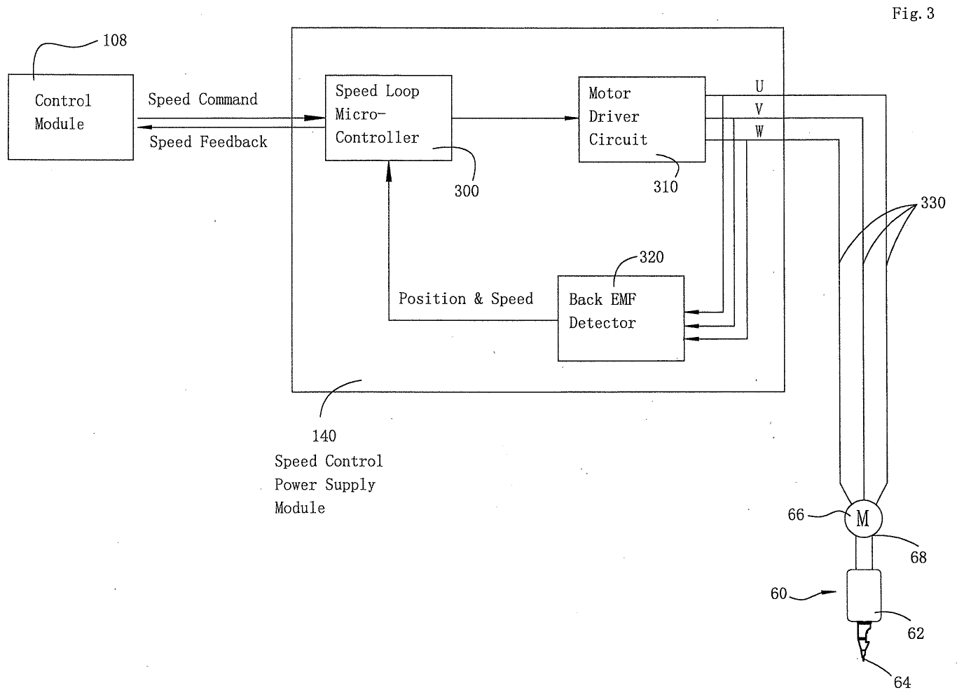

For example, as further illustrated in FIG. 3, power supply 140 may include a controller 300, a motor driver circuit 310, and a back electromotive force (Back EMF, or BEMF) detector 320 for detecting the position and speed of the motor's rotor in tattoo device 60. Controller 300 may be a microcontroller with a closed-loop speed control circuit or chip. Commercially available microchips for BLDC motor control may be used.

As illustrated, the BLDC motor 66 in tattoo device 60 may have three phase windings, and the motor driver circuit 310 and the BEMF detector 320 may also have three corresponding connection wirings 330 (indicated as wires or terminals U, V, and W in FIG. 3 respectively). An example of such a motor is a Delta 3-phase sensorless BLDC motor. The three phases in a three-phase BLDC motor may also be alternatively referred to as A-B-C phases, or R-S-T phases, instead of U-V-W. Typically, the three phases may be out of phase with regarding to one another, such as by 120°, as can be appreciated by those skilled in the art. The BEMF detector 320 may also include a back EMF zero-crossing detection circuit.

Microcontroller 300 may be a special controller adapted for speed control of a BLDC motor, in combination with motor driver circuit 310 and BEMF detector 320. For example, commercially available microcontrollers such as a brushless motor driver integrated circuit available from Toshiba™ under the model number TB6575FNG, or a microchip or microcontroller commercially available under the brand name AVR™ from Atmel or Microchip Technology, or other microcontrollers provided by Microchip Technology such as PIC18FXX31 MCUs or the like, may be used in the speed control power supply module 140.

Motor diver circuit 310 may include a three phase inverter bridge. As can be appreciated by those skilled in the art, a sensorless BLDC motor may have a star winding connection or a three-phase bridge connection. In some embodiments, the three-phase bridge connection may be used, which only require three wiring connections to the motor. In some embodiments, the star winding connection may be used, which may require four wiring connections.

If a BLDC with sensors is used, the BEMF detector 320 is not necessary. For three-phase BLDC, three separate Hall sensors may be used, each for a respective phase. For example, Hall sensors may be mounted in the motor housing and additional wirings may be provided to connect the motor to the control circuit. Hall sensors may send position signals of the rotor to controller 300 through these additional wirings.

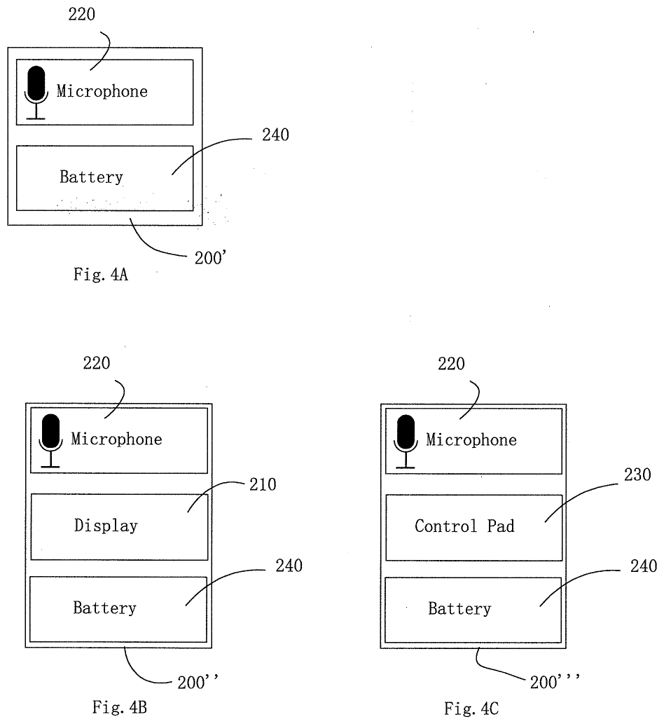

In different embodiments, the portable device 200 may be modified as illustrated in FIGS. 4A, 4B and 4C. In particular, the portable device 200′ shown in FIG. 4A has a microphone 220 and a battery 240, but is not provided with any keypad or display. The portable device 200″ shown in FIG. 4B has a display 210, a microphone 220, and a battery 240, but is not provided with any keypad. The portable device 200′″ shown in FIG. 4C has a microphone 220, a control pad 230 such as a keypad, and a battery 240, but is not provided with any display.

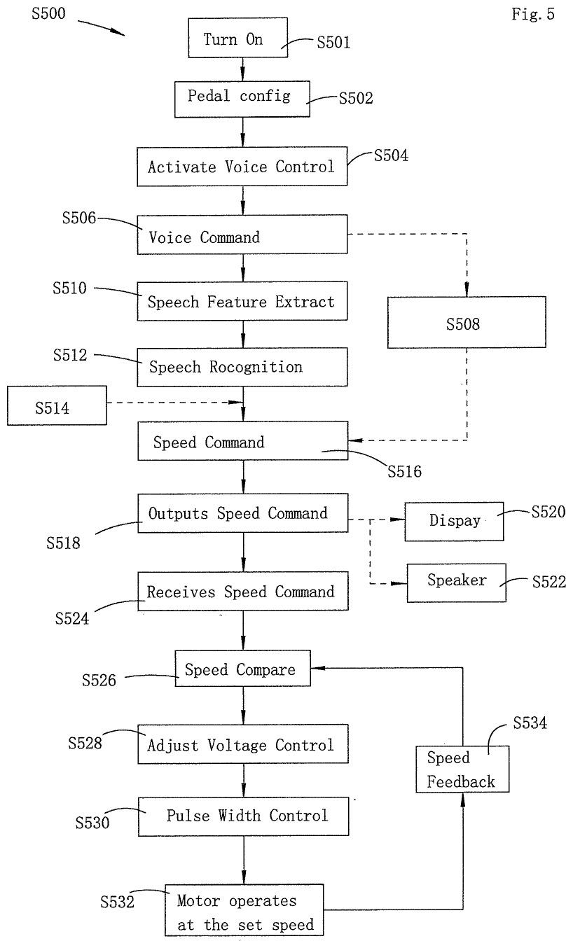

In operation, system 100 or 100′ may be used as follows, as illustrated in FIG. 5, which shows a flowchart for an example process S500.

At S501, system 100 is connected to power source 102 through AC/DC convertor 104 if they are not already connected, and is turned on.

At S502, pedal 46 is configured, such as through control module 108 or portable device 200. Pedal 46 may be configured to serve as an on-off switch for voice control, or configured to provide more complicated control signals as will be further described below.

At S504, voice control may be activated, such as by utterance of a trigger command by the operator, or by pressing pedal 46 if pedal 46 is configured to activate voice control. Alternatively, voice control may be activated by utterance of any voice command, or another device such as the portable device 200.

At S506, a voice command (see below for possible voice commands) is uttered by the operator and a corresponding audio signal may be detected and received by microphone 160 and provided to speech recognition module 130 to parse and analyse the voice command. Speech recognition module 130 can either analyse the received audio signal locally, or send the audio signal or data to a remote speech recognition server such as cloud speech recognition service 900 for analysis at S508.

In any event, the audio signal or data may be analysed to extract words at S510, and the extracted words are constructed to form a corresponding commend, which may be matched with a set of pre-selected commands, through a speech recognition process or algorithm at S512.

Alternatively, control pad 170, which may include a keypad, may be used to enter the command directly at S514.

Control pad 170 may also be used to enter initial or default operation speed value. For example, a keypad in control pad 170 may include keys (not shown) each corresponding to a different speed setting/value. The keys may be labelled by the speed level or value, or may be indicated by the appropriate operation at the corresponding speed. In some cases, a key may be labelled as “Line Drawing” to indicate the speed most appropriate for drawing lines. Another key may be labelled as “Colour Fill”, to indicate the speed for filling colours. A key may be configured to increase the speed by a pre-selected amount and another key may be configured to decrease the speed by the pre-selected amount.

In any event, a command for speed control is received by controller 110 of system 108 at S516.

At S518, control system 110 may provide the command to speed control power supply module 140 or microprocessor 300.

The constructed or received command may be audibly repeated or displayed to the user at S518.

For example, the command may be displayed on display device 180 at S520 and repeated through speaker 190 at S522.

The control command may be performed without confirmation to reduce wait time. Alternatively, a received command, particularly a voice command, may be confirmed by the user before it is executed, in case there is any error in the speech recognition process.

Speed control power supply module 140 receives the command at S524 and processes the command to adjust the speed of the motor 66.

In an example execution, the command may be “100 Hz” or “6000 RPM”, which sets a desired motor speed at 6000 RPM.

At S526, microcontroller 300 may compare the set speed to the current speed of the motor 66, which may be lower or higher than, or at, the set speed. The current speed may be determined based on signal or feedback from BEMF Detector 320. For example, BEMF detector 320 can measure the back-EMF in the three phases U, V and W, which are indicative of the rotor positions in the BLDC motor, and send corresponding data signals to microcontroller 300 for processing and calculating the motor speed.

At S528, the output power (e.g. voltage) at the outlet terminals U, V and W of motor driver circuit 310 are adjusted to adjust the motor speed based on the set speed and the comparison result.

The motor speed may be adjusted in different optional approaches. In one approach, the peak values of the output voltages may be adjusted. In another approach, the output voltages at the output terminals include pulses, and the duty cycle of the output voltages is adjusted by adjusting or modulating the pulse width of the output voltages, in which case the peak voltage values may or may not be adjusted. A duty cycle may be expressed as a fraction, such as a percentage or a ratio, and generally refers to the fraction of time the output voltage is “on” as compared to the fraction of time it is “off” in a cycle. The pulses may have a constant frequency, with the length of each cycle being equal.

Thus, in addition to or instead of voltage adjustment, the motor speed may also be adjusted by pulse width modulation (PWM) speed control at S530. If this control is utilized, the output of power supply 140 may be modulated to provide pulses of the output voltage at different duty cycles. The pulse width modulation of the output voltages at terminals U, V and W of motor driver circuit 310 adjusts the motor speed, and the PWM may be performed based on the set speed and the comparison result to change the motor speed so it approaches and reaches the set value.

If the actual speed of the motor 66 is the same as the set speed, no further action is needed.

If the actual speed is lower than the set value, the motor speed may be increased by increasing the output voltages, or the duty cycle, or both, at terminals U, V and W of motor driver circuit 310.

If the actual speed is higher than the set value, the motor speed may be decreased by decreasing the output voltages, or the duty cycle, or both, at the U, V and W terminals of motor driver circuit 310.

At S532, the motor 66 operates at the speed according to the output from U, V and W terminals.

A speed feedback is provided by BEMF detector 320 at S534, and the next iteration of speed adjustment may be performed if the speed comparison result shows that the actual speed is still not equal to the set speed within any given tolerance. Specifically, the actual motor speed under the current output voltages at U, V and W of the motor driver circuit 310 may be detected by BEMF detector 320, and used in a feedback control loop to further adjust or maintain the current speed by speed loop microcontroller 300. This way, the output voltages or duty cycle of motor driver circuit 310 may be repeatedly or iteratively adjusted based on the feedback motor speed until the set motor speed is achieved within any given tolerance.

In a modified embodiment, a separate foot pedal (not shown) may also be connected directly to power supply 140. This foot pedal may be used as a switch to turn power supply 140 on or off.

The power supply 140 may also include other controls, switches, knobs or other components that allow a user to manually adjust power supply 140 or turn it on or off.

Tattoo device 60 may be connected to power supply 140 via a cable, which may include connection wires for each of the three phase terminals U, V and W, and any suitable connection or coupling structure, such as a terminal or connector.

Power supply 140 may supply modulated and phased DC power to tattoo device 60. For each phase, the DC voltage signal may have a square wave profile, and the three phases may be out of phase by 60 degrees to provide the required power to drive the BLDC motor.

Tattoo device 60 can include any suitable brushless DC motor with variable needle oscillating frequencies. Tattoo device 60 may include needles 64 or a bundle of needles 64 for applying ink to a subject's skin, a base 68 for actuating downward movement of the needle(s) 64, and a needle handle 62 that connects needle(s) 64 (or a needle module) to base 68 and can be held in a hand of the operator during a tattoo session.

In some embodiments, base 68 may also be a conventional rotary tattoo machine, and may include an electric DC motor, which when powered by power supply 140 can generate rotary mechanical motion. Base 68 may also include a transmission mechanism (not separately shown) to convert the rotary mechanical motion to linear motion for driving the reciprocal oscillating motions of the needle(s) 64.

In some embodiments, a power control system disclosed herein may be adapted to work with different types of tattoo devices. For example, the power supply 140 may include a separate power source for supplying a DC current at a variable voltage suitable for operating a regular DC motor, and may control the motor speed based on an open loop control circuit.

In some embodiments, base 68 may alternatively include a coil tattoo machine. In further embodiments, base 68 may include any other type of electrically powered tattoo machine. For example, the power supply 140 may include a power source for supplying a DC current at a variable voltage suitable for operating a regular coil tattoo machine, and may control the motor speed based on a separate open loop control circuit.

The motor of the tattoo machine may have an operational rotation frequency (speed) of 50-200 Hz.

In system 100′, control module 108 may be connected to power supply 140 via a cable and input/output connectors or terminals, or through wireless connections. However, in system 100, it may not be necessary to connect power supply 140 and control module 108 through a separate connection, and the power supply and the control module may be integrated into one unit or parts of the power supply and the control module may be provided on the same circuit board.

The control module 108 or controller 110 may be provided using any suitable components or processors.

For example, controller 110 may include an audio processor, a graphics processor and a general processor (not separately shown).

Controller 110 may include any suitable computer processor such as microprocessor, and may be configured to execute computer readable instructions stored on a memory such as memory 120. For example, controller 110 may be a general purpose processor.

Memory 120 may be a no-volatile computer-readable media. Memory 120 may store thereon computer-readable and executable instructions for performing the operations described herein. Memory 120 may also store a data structure, such as a file or table, including a number of commands for controlling the operation of the tattoo device 60. For example, the commands may include one or more of the following commands:

TABLE I
List of Possible Commands

| Command | Action |
|---|---|
| Start | Start motor |
| Stop | Stop motor |
| GO | Start motor |
| UP/Accelerate | Increase motor speed |
| DOWN/Decelerate | Decrease motor speed |
| Speed 1, 2, 3, . . . | Set the motor speed to the speed corresponding to the |
| | preset setting 1, 2, 3, . . . respectively |
| Turn | Set the motor speed to a lower value suitable for making a |
| | turn in the tattoo |
| Turn 1, 2, 3, . . . | Set the motor speed to a value suitable for the type of turn |
| | represented by the turn type 1, 2, 3, . . . |
| Next Lower Speed | Decrease the motor speed by a predefined increment |
| Next Higher Speed | Increase the motor speed by the predefined increment |
| Max (Speed) | Set the motor speed to the maximum speed |
| Minimum (Speed) | Set the motor speed to the minimum speed |
| 50, 60, . . . 150 (Hz) | Set the motor speed to the identified value |
| Go Back | Set the motor speed to the previous speed value |
| Line | Set the motor speed to a value suitable for drawing a line |
| Straight (Line) | Set the motor speed to a value suitable for drawing a |
| | straight line |
| Curve (Line) | Set the motor speed to a value suitable for drawing a |
| | curve |
| Fill (Color) | Set the motor speed to a value suitable for filling an area |
| Shade | Set the motor speed to a value suitable for drawing a |
| | shade |
| Standard | Set the motor speed to a standard speed |
| Old Style | Set the motor speed suitable for tattooing in the old style |
| Realistic | Set the motor speed to a value suitable for drawing a |
| | realistic tattoo |
| EYEBROW | Set the motor speed suitable for drawing an eyebrow |
| LIPS | Set the motor speed suitable for drawing a lip |
| EYELINER | Set the motor speed suitable for applying an eyeliner |
| Thin Needles | Set the motor speed to a value suitable for tattooing with |
| | thin needles |
| Thick Needles | Set the motor speed to a value suitable for tattooing with |
| | thick needles |
| Mode 1,2, . . . | Operate the motor in the selected mode, e.g. mode 1 or |
| | mode 2. |
| Ink | Stop the motor, or lower the motor speed, for a selected |
| | period of time (e.g. a few seconds) to allow time to load |
| | ink into the ink reservoir(i.e. by dipping the needle module |
| | into an ink bottle) |
| Wash | Intermittent high speed for the purpose of washing the |
| | needle in a washing liquid |
| Lighting | Turn on the illumination light (when such light is provided |
| | in the tattoo machine) |
| Tattooing | Start motor at a preselected initial speed for tattooing |
| Session Time | Display or state the elapsed time for the session |
| Timer On | Set the timer in the controller on |
| Timer Off | Set the timer in the controller off |
| Reset | Reset the control system |
| Power Off | Turn off the power to the system |

As can be appreciated, control module 108 may also be configured to automatically select the suitable or optimal operation frequency based on a combination of the commands listed above. For example, for thin needles, a higher frequency (speed) is usually used, and for thick needles, a lower frequency is usually used. For drawing different lines or different parts of the body, different frequencies may also be selected. The operator or manufacturer of the control system may pre-set the frequency for a given combination of these factors. When the operator utters a command that matches the given combination, the corresponding frequency will be automatically selected. The control algorithm may also gradually adjust the frequency over time for a given combination. This may be more convenient for the operator.

For example, after a Turn 1 command, the control module 100 may adjust the frequency to gradually reduce the frequency for a few seconds (the expected time for the operator to complete the turn), and (after the turn) automatically gradually increase the frequency back to the initial frequency.

The control module 108 may be programmed to handle voice commands uttered in different languages, such as English, French, German, Chinese, Spanish, Italian, or the like. In some embodiments, control module 108 may be programmed to handle multiple languages and include a user interface for a user to select the preferred language.

An audio processor may be a general purpose processor or a special processor configured for processing audio data. Audio processor may include an integrated circuit board adapted to process analog electrical audio data and generate corresponding digital audio data. For example, the processed audio data may be saved in the form of a pulse-code modulation (PCM) data stream, or another suitable format.

Control system 100 also includes input/output (I/O) interfaces for connection with other devices in the system or external devices. The I/O interfaces may be configured to facilitate bidirectional communication, and transfer power, between control system 100 and devices connected to control system 100 through the I/O interfaces. For example, possible I/O interfaces may include a plurality of USB ports, power outlets and inlets, wired and wireless communication ports and devices.

A cable or other connecting components may be used to connect different components and units in the system, such as keypads, pointing devices, microphones or other acoustic sensors, electronic visual displays, speakers, or the like.

Control system 100 may also include input/output devices electrically connected to controller 110, which may be fixedly mounted to, or embedded within, the housing of control module 108. For example, as depicted in FIG. 1, control module 108 may include an electronic visual display 180 mounted onto the same housing has control module 108 for displaying information regarding the operating condition of control system 108 and motor condition of motor 66. Other input/output devices may include keypads, buttons, microphones, knobs, dials, switches, speaker, or other devices for controlling the operation of control system 100 or allowing control module 108 to provide feedback to the operator.

Control module 108 may also include one or more wireless transceivers, such as transceiver 150 for transmitting and receiving wireless signals. A wireless transceiver may allow control module 108 to communicate with connected devices via Bluetooth™, WiFi or any other wireless communication standard.

As depicted in FIGS. 1 and 2, a microphone 160 is provided in the system, which may be used for detecting and receiving voice commands from the operator. In different embodiments, microphone 160 may be connected to control module 108 via one of the input/output interfaces discussed above, or may be embedded or integrated within control module 108. Microphone 160 may be a wireless microphone in wireless communication with control module 108.

Foot pedal 46 may be connected to control module 108 through one of the input/output interfaces. The foot pedal 46 may include electrical circuits for sending control signals to control module 108. In some embodiments, foot pedal 46 may have a wireless transceiver (not separately shown) and a battery 462 and may be wirelessly connected to control module 108.

In some embodiments, foot pedal 46 may be configured as a simple on-off switch. For example, when the foot pedal 46 is pressed by a foot, the switch is “on” in the sense that the voice control system is activated and can detect and process a voice command. When the foot pedal is released, the switch is “off” and the voice control system is inactive so the operator and customers can carry on a conversation without the risk of unintentionally triggering the voice control system and leading to unintended consequences.

In different embodiments, the switch may be a toggle switch and can be turned on or off by pressing and releasing the foot pedal once.

In an embodiment, power supply 140′ is configured to receive control signals from control module 108 and adjust or set the output voltage and duty cycle in response to the received control signal. Optionally, power supply 140 may include a voltage adjustment interface (not separately shown) to allow an operator or user to manually adjust the output voltage or duty cycle of power supply 140. The interface may include a dial, knob, keypad or other physical structures that allow a user to adjust the power supply 140.

In some embodiments, power supply 140 may also include its own display (not shown) for displaying the output voltage, duty cycle, or corresponding motor speed/frequency in real time. Power supply 140 may include an AC to DC converter, which converts mains electricity to low voltage DC output, or a DC to DC power regulator. For example, power supply 140 may be a switch mode power supply (SMPS) and may convert input AC power of 120 V and 60 Hz to output (DC) power, which may have a three-phase square waveform with peak voltage of about 4 V to about 18 V. The output of power supply 140 may be relatively stable, or may vary with time. For example, power supply 140 may output a three-phase square wave pulse-width modulated (PWM) voltage or electricity power.

The motor of the tattoo machine may have an operational rotation frequency (speed) of 50-200 Hz.

In an embodiment of another process, an operator may connect power supply 140 to an electricity source, such as mains power. Tattoo device 60 may be turned on with its own independent power switch if provided. Once power supply 140 is provided with power and is turned on, it is ready to output power to tattoo device 60 subject to control by control module 108. Control module 108 may also receive power from power supply 140 or from another power source, and may be turned on when power supply 140 is turned on. Tattoo device may be operated with the control module 108 deactivated or activated. That is, it is possible to bypass the control module 108 if the operator chooses to do so. The following description presumes however that the control module 108 is activated and will be used to control the tattoo device 60 by the operator.

Referring back to FIG. 5, the operator may activate microphone 160 at S504 by depressing foot pedal 46. For example, microphone 160 may be configured to be activated while pressure is being applied to foot pedal 46 (e.g. by operator stepping on foot pedal 46). Alternatively, microphone 160 may be configured to be activated when pressure is applied to foot pedal 46 and then removed (e.g. by the operator tapping foot pedal 46).

Alternatively, microphone 160 may be always on and is ready to detect any triggering voice command.

At S506, the operator utters a voice command, which is received by microphone 160. The microphone 160 converts the sound signal associated with the voice command into an electric signal to be processed by speech recognition module 130 and controller 110.

The operator may deactivate microphone 160 after uttering the voice command, for example, by removing pressure from foot pedal 46, or by pressing and releasing foot pedal 46. Advantageously, by deactivating microphone 46 after providing the voice command, the operator can prevent control module 108 from inadvertently responding to environment sounds such as conversations between the operator and the tattoo subject or others. For example, by deactivating microphone 160 or control module 108, the operator can prevent control module 108 from interpreting background noise or other sounds made by a human as voice commands. Microphone 160 may alternatively be automatically deactivated after a time period of inactivity.

Optionally, at S508, the received voice signal may be sent to a cloud speech recognition service such as server 900 for processing, to extract the voice command. The voice command is then received by control system 108 or controller 110 at S516.

Alternatively, at S510, speech recognition module 130, which may include an audio processor (not shown), processes the electrical signal from microphone 160 and converts it into a digital audio data, which is then processed to extract the voice command. For example, an example method of extracting speech features may be based on an auditory model.

At S512, the digital audio data with speech feature parameters is parsed and matched to one or more of pre-defined instructions stored on memory 120, and a control signal corresponding to the selected instruction is generated by controller 110 at S516. One or more digital audio data with speech feature parameter patterns may be matched to a particular one of the stored instructions. Multiple instructions may correspond to a particular control signal for controlling power supply 140. In some embodiments, speech recognition module 130 may process the audio data and extract the speech features and store the extracted text as a text file, and transmit the text data or text file to controller 110 for further action. In some embodiments, speech recognition module 130 may compare and match the received audio signal directly with audio signals stored in a database of known voice command audio signals, and transmit the comparison or matching results to controller 110. Controller 110 can then process the received signals or results and send the corresponding voice command(s) for speed control to speed loop microcontroller 300 at S518.

For example, the operator may turn on the tattoo device by providing the voice commands “DEVICE ON”, “TURN ON TATTOO MACHINE” or another voice command. The operator may set the operating frequency of tattoo device 60 to 150 Hz by uttering the voice commands “150 HERTZ”, or “HIGH SPEED”, or other voice command to a similar effect. The operator may also specify the output voltages of the variable power supply 140. Since the operating frequency of tattoo device 60 is a function of the voltage or voltages supplied to the tattoo device 60, in some situations, the operator may indirectly set the operating frequency of the tattoo device 60 by specifying the output voltage of the variable power supply 140. For example, this control method may be used with an open loop speed control system. However, in most situations, it may be more convenient to be able to set the actual motor speed or frequency by stating the desired speed or frequency. For example, with a closed-loop speed control and a BLDC motor, particularly a sensorless BLDC motor, the motor speed may be accurately determined and controlled, and the speed control may be carried out by directly setting the desired speed value.

The set of acceptable instructions may be changed or modified by the operator. For example, the operator or another user may re-configure or re-program the instructions stored on memory 120 to add additional acceptable instructions through the use of any suitable input device connected to control module 108.

At S518, the control signal may be transmitted to power supply 140 to control the power output from power supply 140 to tattoo device 60. For example, the power supply 140 may be controlled to alter the voltage or duty cycle or both of its power output in response to the received control signal. The operating frequency of tattoo device 60 then changes in response to the change in the input voltage and duty cycle. With a BLDC motor, the voltage or the duty cycle of the input at the respective U, V, W connections may be adjusted to control the motor speed.

After the operating frequency of tattoo device 60 has been changed as described above, control module 108 may repeat the process from S504 or S514, and is ready to receive further input from the operator.

After a frequency change, the motor speed may be measured and controlled with a closed-loop speed control algorithm, and the actual speed value or corresponding frequency may be displayed or reported audibly through the speaker.

When desired, the operator may deactivate or pause operation of tattoo device 60, for example, by providing a voice command as set out above, or manipulating other controls on tattoo device 60, power supply 140 or control module 108.

During operation of system 100 or 100′, information on the operating conditions of the control module 108, the power supply 140 and the tattoo device 60 may be presented to the operator. For example, the operating frequency or motor speed of the tattoo device 60 may be displayed to the operator on an electronic visual display 180 of the control module 108 or display 210 on the portable device 200 when it is used. Additionally and alternatively, the operating frequency or motor speed may also be audibly reported, such as through the speaker 190 of control module 108 or portable device 200 if it includes a built-in speaker.

In an alternative embodiment of the voice control system, a foot pedal may be omitted and the operating frequency of tattoo device 60 can be controlled solely through verbal commands.

In this embodiment, a voice activation mechanism may still be provided in the voice control system. For example, a voice based activation mechanism may be provided to activate the voice control system when a pre-selected trigger or activation command is detected by the microphone 160.

In some embodiments, control module 108 may be configured for wireless communication with a portable or mobile input/output device such as a smart watch. The smart watch may include an input interface and input devices such as microphone, a physical keypad or on-screen soft keypad, and may include output devices such as a speaker or headphone outlet, and a display screen. For use with such a portable device, a disposable pouch may be provided.

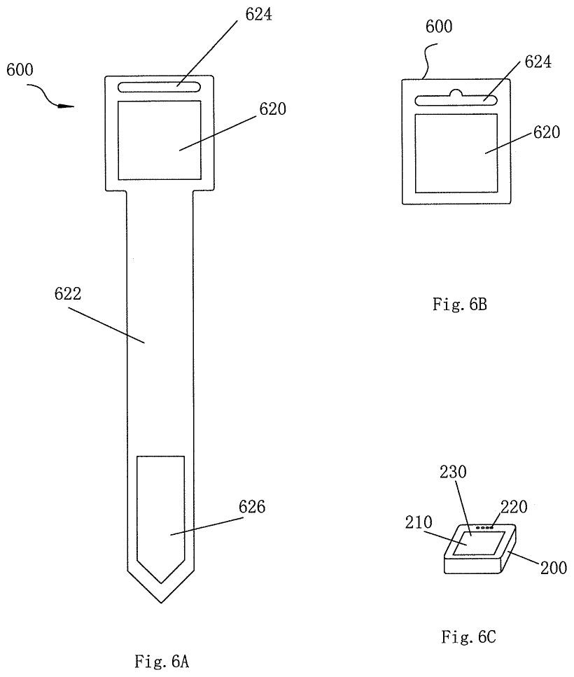

As illustrated in FIGS. 6A and 6B, a pouch 600 may be formed of a soft material and have a pocket 620 sized to store the mobile device 200, which may be shaped as illustrated in FIG. 6C. As illustrated in FIG. 6A, Pouch 600 may have a band 622 attached thereto and have an opening 624 to allow pouch 600 to be conveniently attached to the operator's arm or wrist. Optionally, the terminal end of band 622 may be provided with a self-sticking material 626 for easy attachment. The pocket 620 may be made of a transparent material to allow the user to see the display 210 on the mobile device 200 when it is stored in the pocket 620. Pouch 600 is convenient to use as it allows the mobile device 200 be used without the risk of contamination and the need to clean the mobile device 200 after use. The pouch 600 containing the mobile device 200 may be conveniently placed near the operator and be attached to a place near the operator, such as a chair, or attached to a part of the operator's body such as an arm, or even a body part of the subject being tattooed. It is also possible to attach the pouch 600 to the tattoo device 60, or a cable connected to the tattoo device 60. The pouch may be optionally provided with different attachment mechanisms such as a sticky material, a hook, a string or an opening for hanging the pouch in a hook or the like. The pouch 600 may be made from a transparent plastic film. The pouch 600 may be manufactured for a single-use. During use, the operator or user can touch the external surface of pouch 600 to operate device 200 without coming into direct contact with the device 200. Thus, the operator can use device 200 to control the operating or motor speed without contaminating mobile device 200, thus avoiding or reducing the risk of cross-contamination.

In a process of using a control system without a foot pedal, the following actions may be performed.

Operator initially connects power supply 140 to power source 102 and turns the power supply 140 on. Tattoo device 60, control module 108 and microphone 160 are also turned on.

The operator 80 utters a voice command, which may initially be a trigger command, and the audio signal is detected by microphone 160 and converted into an electronic signal by speech recognition module 130.

Optionally, when a voice activation mechanism is provided, the control module 108 is first activated by a trigger or activation command before a function voice command is uttered. The control system 100 then processes the electrical signal as described above and controls the motor speed accordingly.

To prevent tattoo device 60 from inadvertently changing its operating state in response to background noise or other audio, the control system 100 may store a trigger or activation command in memory 106. The trigger or activation command may include one or more words or phrases used by the operator to indicate an intention to control tattoo device 60 using the voice control system. The trigger word may be any word as long as the operator is aware of the word and the trigger word is registered in the control system. Before the trigger word is uttered and detected, or after a pre-defined period of silence after detecting a trigger word, control module 108 is set in a standby mode or state. In the stand-by mode, control module 108 will not attempt to parse sounds detected by microphone 160 to determine if they match any functional commands, except for recognizing the trigger word. Upon detecting the trigger word, control module 108 will process the audio data received immediately following the trigger word, or within a pre-defined short period, to determine if the uttered sound matches any command(s) stored on memory 120. Control module 108 may return to its standby mode after the audio data has been detected that matches one more instructions stored in memory 120 and a corresponding control signal has been sent to variable power supply 140. Control module 108 may also return to its standby mode upon the detection of another trigger word, or after a predetermined amount of time has elapsed after it has exited its standby mode.

Memory 120 may also store instructions programmed with a voice print recognition algorithm, which, when run by speech recognition module 130 and controller 110, causes control module 108 to accept voice commands only from one or more accepted users. For example, control module 108 may be programmed to recognize the voice of a particular operator, and may thus only generate control signals to change the power output of power supply 140 when an acceptable instruction is received from the particular operator.

Optionally, the voice control system 100 may be trained by a particular operator before use for better recognition, faster and more accurate parsing of the voice commands uttered by the operator. Conventional voice training algorithms and technics known in the art may be used for this purpose. Control module 108 or speech recognition module 130 may be trained to better recognize the words and sentences uttered by the particular operator, according to suitable techniques including those known to the skilled persons in the art.

Controller 110 may also be programmed to filter out background noise from microphone 160 and accept voice commands only when the detected sound signal is above a certain intensity threshold. For example, processor 104 may only parse voice commands when the sound received by microphone 160 has intensity above about 60 decibels. For this reason, the microphone 160 may be conveniently placed close to the operator's mouth or head. For example, it may be included in a headset worn by the operator.

Control system 100 may be configured to inform the operator about the operating state of control module 108 and tattoo device 60. For example, displays or speakers connected to control module 108 may alert the operator when the control module 108 is prepared to receive verbal commands, or when a verbal command provided by the operator does not correspond to any of the instructions stored in memory 120. As depicted in FIG. 1, control module 108 may be configured to display the operating condition of control system 100 on display 180.

When an acceptable command/instruction has been detected, the control module 108 sends a control signal to power supply 140 to control the operating frequency of tattoo device 60 as described above. After the operating frequency of tattoo device 60 has been changed as described above, control module 108 may be ready to receive further input or command from the operator.

In some embodiments, a foot pedal may be used to activate or deactivate the motor in the tattoo device, such as by turning it on or off depending on the state of the pedal. In some embodiments, the control system may be configured, such as by programing the controller 110, to toggle the operation mode of the foot pedal. The foot pedal may be operable in a first mode to activate and deactivate the microphone, and hence the voice commend detection, and in a second mode to activate and deactivate the needle actuator (motor 66).

In different embodiments, speech recognition of the voice commends may be processed locally, or remotely through a remote server such as a server located in a network (cloud server). In some embodiments, the detected audio signal or data may be provided for both local and remote processing, and the first returned result is used for controlling the motor speed, or the two results are compared for confirmation.

In some applications, the motor speed control techniques disclosed in U.S. Pat. Nos. 5,789,895, 7,122,985, or U.S. Pat. No. 6,686,714 may be adopted or adapted for use in an embodiment disclosed herein. In some embodiments, a sensorless brushless motor is used. The power supply to the motor may be connected with three phase Delta wiring, in which case the cable connecting the control module or power supply to the motor of the hand-held tattoo device only needs to accommodate three electrical power wires.

### CONCLUDING REMARKS

It will be understood that any range of values herein is intended to specifically include any intermediate value or sub-range within the given range, and all such intermediate values and sub-ranges are individually and specifically disclosed.

It will also be understood that the word “a” or “an” is intended to mean “one or more” or “at least one”, and any singular form is intended to include plurals herein.

It will be further understood that the term “comprise”, including any variation thereof, is intended to be open-ended and means “include, but not limited to,” unless otherwise specifically indicated to the contrary.

When a list of items is given herein with an “or” before the last item, any one of the listed items or any suitable combination of two or more of the listed items may be selected and used.

Of course, the above described embodiments of the present disclosure are intended to be illustrative only and in no way limiting. The described embodiments are susceptible to many modifications of form, arrangement of parts, details and order of operation. The invention, rather, is intended to encompass all such modification within its scope, as defined by the claims.

What is claimed is:

1. A power supply and control system for a tattoo machine, wherein the tattoo machine comprises an electrical motor for actuating a needle attached to the machine, the system comprising:
a power supply for supplying power to drive a commutatorless direct-current motor, the power supply comprising a motor driver for providing a variable power output and a connector for connection with the commutatorless direct-current motor to apply the variable power output to the motor to adjust a speed of the motor;
a controller integrated with or connected to the power supply for controlling the power supplied to the motor to control the speed of the motor, wherein the controller comprises
a microphone for detecting a voice command, and
a processor programmed to process the voice command and provide an input to the motor driver to control the power output of the motor driver based on the voice command; and
a foot pedal comprising a switch connected to the controller, the switch switchable between an on-state and an off-state when the foot pedal is pressed by a foot, wherein the controller is configured and operable to receive and respond to the voice command when the switch is in the on-state, but to ignore the voice command when the switch is in the off-state.

2. The power supply and control system of claim 1, wherein the commutatorless direct-current motor is a sensorless motor and the motor driver comprises a closed-loop speed control circuit.

3. The power supply and control system of claim 1, further comprising a cable for connecting the motor driver to the motor, wherein the motor comprises three windings, the motor driver comprises at least three output connectors, and the cable comprises at least three wires for connecting respective ones of the at least three output connectors of the motor driver to corresponding ones of the three windings of the motor.

4. The power supply and control system of claim 1, wherein the switch is a toggle switch.

5. The power supply and control system of claim 1, wherein the controller comprises a speech recognition chip configured to perform speech recognition locally.

6. The power supply and control system claim 1, wherein the processor is programed to control one or more operation parameters of the tattoo machine, and the voice command comprises a command to adjust or set each one of the one or more operation parameters, the one or more operation parameters comprising the speed of the motor.

7. The power supply and control system of claim 6, wherein the processor is programed to adjust or set a plurality of operation parameters of the motor in a time period in response to a single voice command.

8. The power supply and control system of claim 1, wherein the voice command comprises a command to increase the speed of the motor, a command to decrease the speed, and a command to set the speed at a value represented by the voice command.

9. The power supply and control system of claim 1, wherein the controller comprises a processor-readable storage media storing thereon a plurality of pre-defined commands, and the controller is configured to determine if the voice command matches any of the stored commands and to execute the voice command in response to determining a match.

10. The power supply and control system of claim 1, wherein the foot pedal is further configured to selectively activate or deactivate the tattoo machine or the motor.

11. The power supply and control system of claim 1, wherein the controller is programmed to select an operation mode of the foot pedal, wherein the foot pedal is operable in a first mode to activate and deactivate detection of the voice command, and operable in a second mode to selectively activate and deactivate the tattoo machine or the motor.

12. The power supply and control system of claim 1, wherein the controller is configured to communicate wirelessly with a portable or mobile input device.

13. The power supply and control system of claim 12, wherein the portable or mobile input device is a wearable device.

14. The power supply and control system of claim 12, wherein the mobile input device comprises a microphone or a smart watch.

15. The power supply and control system of claim 12, wherein the mobile input device comprises a display and an input interface.

16. A tattoo machine comprising a needle actuator comprising a commutatorless direct-current motor, and the power supply and control system of claim 1 operably connected to the needle actuator.

17. A tattoo device comprising:
a needle actuator comprising a commutatorless direct-current motor; and
a controller connected for controlling operation of the needle actuator, wherein the controller comprises
a motor driver connected to the motor for measuring and controlling a speed of the motor;
an acoustic sensor for detecting voice commands uttered by a user, wherein the voice commands comprise a command to set the speed of the motor at a value indicated by the command, and
a processor programmed to process received voice commands and provide an input to the motor driver to control operation of the needle actuator based on the received voice commands,
wherein the controller is configured and operable to respond to the command to set the speed of the motor at the value indicated by the command.

18. The tattoo device of claim 18, wherein the needle actuator comprises a sensorless motor, and the motor driver comprises a closed-loop speed control circuit.

19. The tattoo device of claim 17, wherein the controller comprises a speech recognition chip configured to perform speech recognition locally.
